(19) United States
(12) Patent Application Publication
(10) Pub. No.: US 2015/0240272 A1
(43) Pub. Date: Aug. 27, 2015

(54) LACTATE PRODUCTION PROCESS

(71) Applicant: PLAXICA LIMITED, London (GB)

(72) Inventors: Stephen Alexander Calder Smith, London (GB); James Andrew Surman, London (GB)

(21) Appl. No.: 14/429,245

(22) PCT Filed: Sep. 19, 2013
(86) PCT No.: PCT/GB2013/052450

§ 371 Date: Mar. 18, 2015

(30) Foreign Application Priority Data
Sep. 19, 2012 (GB) 1216716.9
Jan. 11, 2013 (GB) 1300523.6

(51) Int. Cl. C12P 7/62; C08G 63/06; C12P 7/56

(52) U.S. Cl. CPC C12P 7/625; C12P 7/56; C12P 7/62; C08G 63/06

(57) ABSTRACT

A process for producing a compound of formula (I) is provided. The process comprises reacting a mixture comprising a compound of formula (IIA) and a compound of formula (IIB) with an alkyl alcohol in the presence of a Candida antarctica lipase B enzyme to produce a product comprising the compound of formula (I) and the compound of formula (IIB); wherein R¹ is C₁ to C₆ alkyl; and wherein R² is II or C₁ to C₁₂ alkyl. Also provided are processes for the production of R-lactic acid, oligomeric R-lactic acid, R,R-lactide, poly-R-lactic acid, alkyl S-lactate, S-lactic acid, oligomeric S-lactic acid, S,S-lactide, poly S-lactic acid and stereocomplex polylactic acid.

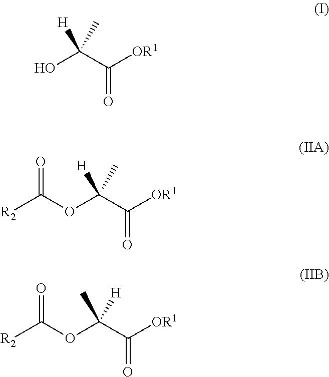

### FIELD OF THE INVENTION

The present invention relates to the production of single enantiomers of lactic acid and its derivatives via a process involving the enzyme-catalysed stereoselective deacylation of O-acyl alkyl lactate. The invention also relates to the subsequent generation of racemic O-acyl alkyl lactate, facilitating the recycling of lactate-containing byproducts generated by the stereoselective deacylation process.

### BACKGROUND OF THE INVENTION

Lactic acid (2-hydroxypropanoic acid) and its cyclic dimer lactide (3,6-dimethyl-1,4-dioxan-2,5-dione) are important building blocks for the chemical and pharmaceutical industries. One example of their use is in the manufacture of polylactic acid, a polymer whose ability to be produced from a variety of renewable feedstocks makes it an attractive candidate to replace more conventional petrochemical polymers. Polylactic acid can be prepared via polymerisation of lactide, in which case polymerisation is typically carried out under carefully controlled conditions to ensure that long polymer chains are produced in preference to shorter oligomers. Alternatively polylactic acid can be prepared directly from the condensation polymerisation of lactic acid, though obtaining high molecular weight material is generally more difficult than via ring-opening polymerisation of lactide.

Today virtually all large scale production of the lactic acid available commercially is carried out by fermentation processes; see for example Strategic Analysis of the Worldwide Market for Biorenewable Chemicals M2F2-39, Frost and Sullivan, 2009. Lactic acid is chiral and can be made in two enantiomeric forms, respectively L-lactic acid (hereinafter referred to as S-lactic acid) on the one hand and D-lactic acid (hereinafter R-lactic acid) on the other. Conventional fermentation technologies principally generate S-lactic acid with little R-lactic acid being formed. Although alternative bacterial strains, often genetically engineered, can be used to produce R-lactic acid, to date the modified bacteria and the associated processes are expensive and difficult to use reliably on a large industrial scale. This is evidenced in the comparatively high price and limited availability of R-lactic acid.

Since the most readily available source of lactic acid is S-lactic acid, the principal lactide employed commercially to date has been S,S-lactide and the polymer produced poly-L-lactic acid (PLLA) (hereinafter poly S-lactic acid). However the physical and chemical properties of poly-S-lactic acid are limited relative to conventional polymers, as are those of the corresponding poly-D-lactic acid (PDLA) (hereinafter poly R-lactic acid), particularly with respect to more durable and/or engineering applications.

It has been found that deficiencies can be overcome by using mixtures of poly S-lactic acid and poly R-lactic acid which are prepared by, for example, melt blending. It is believed that in these so-called ‘stereocomplex’ polymer mixtures close packing of the poly S-lactic acid and poly R-lactic acid chains occasioned by their differing chirality improves polymer crystallinity which leads to improvements in for example, thermal stability. This permits the use of stereocomplex polylactic acid for a much wider range of consumer durable applications, e.g. fibres, making it a viable alternative to traditional commodity polymers such as polyethylene terephthalate, polypropylene and polystyrene. This approach however requires access to large quantities of poly R-lactic acid and therefore ultimately to large quantities of R-lactic acid, or to suitable derivatives thereof. A process which permits the efficient production of such materials would be desirable.

Jeon et at (Tetrahedron Letters, 47, (2006), 6517-6520) disclose the laboratory observation that rac-lactide can be alcoholised with various alcohols in the presence of a solvent and the supported lipase enzyme Novozym-435, to produce a mixture containing the corresponding alkyl R-lactate and alkyl S,S-lactyllactate. However, that document does not deal with the problem of providing an efficient and economic process for producing R-lactic acid-based materials on an industrial scale. Production of the rac-lactide starting material typically involves a multi-step process in which rac-lactic acid is first dehydrated to form lactic acid oligomers, and the oligomers are then depolymerised in the presence of a transesterification catalyst such as tin(II) bis(octanoate) to produce lactide. A mixture of the stereoisomeric forms of lactide (R,R-lactide, S,S-lactide and R,S-lactide), is produced, and so the R,S-lactide must be separated (e.g. by distillation and/or crystallisation) from the S,S-lactide and R,R-lactide to provide the starting material.

Lee et al (Organic Process Research & Development, 2004, 8, p 948-951) discloses a different approach for obtaining R-lactic acid derivatives, in which racemic alkyl lactate is stereoselectively acylated in the presence of Novozym-435 using a vinyl alkanoate. However, the process described in the Lee document leads to the production of an aldehyde byproduct, ethanal, which may decrease the activity/reduce the lifetime of the enzyme. Conversion of the ethanal into vinyl alkanoate (e.g. for reuse in the process), is unlikely to be economically viable at large scale, and so the Lee process is also unlikely to be amenable to industrial scale production of R-lactic acid based products. In fact, both the Jeon document and the Lee document are silent regarding how unwanted byproducts produced by the respective processes may be utilised.

Kazlauskas et al (J. Org. Chem, 1991, 56, p 2656-2665) discloses a process for enantioselective hydrolysis of esters of lactyl acetate. However, whilst Pseudomonas cepacia lipase was found to be effective in catalysing enantioselective hydrolysis of the tert-butyl ester of lactyl acetate in high enantiomeric excess, the other enzymes tested were ineffective.

The present inventors have surprisingly found that O-acyl alkyl lactate (also known as alkyl α-acyloxypropionate) can be stereoselectively deacylated in high enantiomeric excess and high yield by reaction with an alkyl alcohol in the presence of Candida antarctica lipase B. This new process provides products containing the R-lactic acid building block and is particularly suitable for use at industrial scale. In addition, the process does not require the use of rac-lactide as a starting material, does not result in the production of aldehyde byproducts, and facilitates recovery and re-use of valuable lactic acid-containing and other byproducts.

### SUMMARY OF THE INVENTION

The present invention provides a process for producing a compound of formula (I)

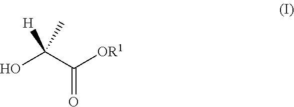

comprising reacting a mixture comprising a compound of formula (IIA) and a compound of formula (IIB)

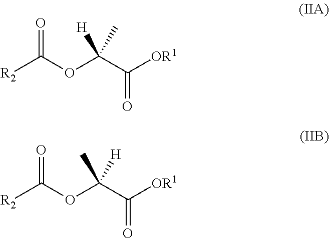

with an alkyl alcohol in the presence of a Candida antarctica lipase B enzyme to produce a product comprising the compound of formula (I) and the compound of formula (IIB);
wherein R¹ is C₁ to C₆ alkyl; and wherein R² is H or C₁ to C₁₂ alkyl.

### DETAILED DESCRIPTION

The invention provides a reproducible and scaleable process which provides lactic acid derivatives in high enantiomeric purity and high yield. Preferably the compound of formula (I) (i.e. alkyl R-lactate) produced by the process has an enantiomeric excess of at least 90%, more preferably at least 95%, still more preferably at least 98%, most preferably at least 99%. Preferably the compound of formula (IIB) produced by the process (i.e. (S)—O-acyl alkyl lactate) has an enantiomeric excess of at least 90%, more preferably at least 95%, still more preferably at least 98%, most preferably at least 99%.

Typically the reaction is run to completion or almost to completion (i.e. such that reaction mixture contains the compound of formula (I) and the compound of formula (IIB) as the major products, together with only small quantities of the compound of formula (IIA). Preferably, the reaction is run so that the compound of formula (IIA) forms less than 10 mol % of the mixture of lactate-containing species (i.e. the mixture of compounds of formula (I), (IIA) and (IIB)), more preferably less than 5 mol %, still more preferably less than 2 mol %, yet more preferably less than 1 mol %, most preferably less than 0.5 mol %. However, the product mixture may also contain greater amounts of the compound of formula (IIA), for example where the end product is intended for use in applications in which S-lactate-containing compounds having lower levels of enantiomeric purity are tolerated. Higher levels of enantiomeric purity in product streams containing the compound of formula (IIB) may be accomplished by, for example, using excess alkyl alcohol to increase the conversion of (IIA) to (I), or by reacting a product mixture (obtained from reaction of the mixture of (IIA) and (IIB) with alkyl alcohol in the presence of enzyme) with alkyl alcohol and enzyme for a further period or periods of time.

The mixture of the compound of formula (IIA) (i.e. (R)—O-acyl alkyl lactate) and the compound of formula (IIB) (i.e. (S)—O-acyl alkyl lactate) used in the process of the invention may be racemic or scalemic. In one embodiment the mixture of the compound of formula (IIA) and the compound of formula (IIB) is racemic. In another embodiment, the mixture is scalemic. The process is typically carried out using a mixture of the compounds of formula (IIA) and (IIB) having a molar ratio in the range of from 20:1 to 1:20, preferably 10:1 to 1:10, more preferably 5:1 to 1:5, still more preferably 2:1 to 1:2, most preferably 1.5:1 to 1:1.5.

The mixture of compounds of formula (IIA) and (IIB) may for example be produced by reacting a mixture of alkyl R-lactate and alkyl S-lactate with a carboxylic acid, preferably in the presence of an acid catalyst, such as Amberlyst 15 hydrogen form. Other methods for producing the mixture of compounds of formula (IIA) and (IIB) include reacting a mixture of alkyl R-lactate and alkyl S-lactate with a carboxylic acid anhydride (e.g. acetic anhydride) or an acid chloride (e.g. acetyl chloride). The mixture may also be produced by esterification of a mixture of α-acyl-R-oxypropionic acid and α-acyl-S-oxypropionic acid.

R¹ is a C₁ to C₆ alkyl group, such as a methyl, ethyl, n-propyl, i-propyl, n-butyl, n-pentyl or n-hexyl group. More preferably R¹ is a C₂ to C₄ alkyl group, such as an ethyl, n-propyl, i-propyl or n-butyl group. Still more preferably R¹ is n-propyl, i-propyl or n-butyl, yet more preferably n-propyl or n-butyl, most preferably n-butyl. In some preferred embodiments, R¹ is a straight chain alkyl group.

R² is H, or a C₁ to C₁₂ alkyl group, preferably a C₁ to C₁₂ alkyl group, more preferably a C₁ to C₄ alkyl group. In certain preferred embodiments, R² is a C₁ alkyl (methyl), a C₂ alkyl (ethyl), or a C₃ alkyl (e.g. n-propyl or i-propyl) group. In certain preferred embodiments, R² is ethyl or n-propyl.

Preferably R¹ is selected from the group consisting of n-propyl, i-propyl and n-butyl, and R² is selected from the group consisting of methyl, ethyl, n-propyl and i-propyl. More preferably R¹ is selected from the group consisting of n-propyl and n-butyl, and R² is selected from the group consisting of methyl, ethyl, n-propyl and i-propyl. Most preferably R¹ is n-butyl and R² is ethyl or n-propyl.

The alkyl alcohol is typically a C₁ to C₆ alkyl alcohol, such as methanol, ethanol, n-propanol, i-propanol, n-butanol, n-pentanol or n-hexanol. More preferably, the alkyl alcohol is a C₂ to C₄ alkyl alcohol, such as ethanol, n-propanol, i-propanol or n-butanol. Still more preferably the alkyl alcohol is n-propanol, i-propanol or n-butanol, yet more preferably n-propanol or n-butanol, most preferably n-butanol. In some preferred embodiments, the alkyl alcohol is a straight chain alkyl alcohol.

Preferably R¹ is selected from the group consisting of n-propyl, i-propyl and n-butyl, R² is selected from the group consisting of methyl, ethyl, n-propyl and i-propyl, and the alkyl alcohol is selected from the group consisting of n-propanol, i-propanol and n-butanol. More preferably R¹ is selected from the group consisting of n-propyl and n-butyl, R² is selected from the group consisting of methyl, ethyl, n-propyl and i-propyl, and the alkyl alcohol is selected from the group consisting of n-propanol and n-butanol. Most preferably R¹ is n-butyl and R² is ethyl or n-propyl, and the alkyl alcohol is n-butanol. The reaction is typically carried out using an alkyl alcohol having an alkyl group which is the same as the R¹ group present in the compounds of formula (IIA) and (IIB), to avoid the possibility of producing a product which contains a mixture of compounds of formula (I) having different R¹ groups. In other words, in those embodiments the alkyl alcohol has the formula R¹—OH, and the alkyl alcohol R¹ group is the same as the R¹ group in the compounds of formula (IIA) and (IIB).

The process may be carried out using the alkyl alcohol as solvent in which case it is preferred that it is chosen so that the mixture of compounds of formula (IIA) and (IIB) is completely or at least partially miscible therewith. Thus, in one preferred embodiment the process is carried out in the substantial absence of added solvent other than alkyl alcohol (not excluding the possibility that the alkyl alcohol, compounds of formula (IIA) and (IIB), and enzyme may contain some residual solvent, such as water). In other embodiments, other solvent may be present in addition to the alkyl alcohol (e.g. a co-solvent), for example a solvent/co-solvent that is miscible with the alkyl alcohol. Typical examples of preferred solvents/co-solvents include non-polar solvents, e.g. hydrocarbon solvents such as toluene, hexane and heptane. Those solvents provide for good stability of the enzyme under operational conditions, and provide for long enzyme lifetime. Other examples of solvents/cosolvents include unreactive oxygen containing solvents for example dialkyl ethers (e.g. diethyl ether, dipropyl ether or MTBE), tetrahydrofuran, 1,4-dioxane, glycol ethers, polyalkylene glycol ethers and the like. Ketone solvents/co-solvents, such as methyl ethyl ketone, methyl isobutyl ketone and acetone, may also be used. The alkyl alcohol or the alkyl alcohol/co-solvent mixture may contain some water. Typically, the alkyl alcohol or alkyl alcohol/co-solvent mixture employed contains less than 1%, preferably less than 0.5% by weight water, to ensure that the enzyme performs optimally. In some preferred embodiments, molecular sieves are used in the process.

The process may be conducted using excess alkyl alcohol with no additional solvent/co-solvent. The process may also be carried out using stoicheometric quantities of alkyl alcohol (in relation to the compound of formula (IIA), or even sub-stoicheometric quantities of alkyl alcohol. In some preferred embodiments, the molar ratio of alkyl alcohol to the compound of formula (IIA) that is input into the process is in the range of from 0.8:1 to 10:1, more preferably from 0.8:1 to 6:1, still more preferably from 0.8:1 to 4:1, most preferably from 1:1 to 2:1.

The process of the invention involves the enzyme-catalysed stereoselective deacylation of (R)—O-acyl alkyl lactate (in preference to (S)—O-acyl alkyl lactate) with alkyl alcohol to produce alkyl R-lactate. The Candida antarctica lipase B enzyme is preferably chemically or physically immobilised on a porous support (e.g. chemically or physically bound to micro or nano beads made of a polymer resin, for example a functionalised styrene/divinylbenzene copolymer or a polyacrylate resin, as is the case for the commercially available material Novozym-435. In one preferred embodiment, the enzyme is Novozym-435. Other preferred enzymes include IMMCALB-T2-150, an immobilised lipase B from Candida antarctica covalently attached to dry acrylic beads manufactured by Chiralvision; IMNICALBY-T2-150, a generic lipase from Candida antarctica lipase B covalently attached to dry acrylic beads manufactured by Chiralvision; IMNICALB-T1-350, a lipase B from Candida antarctica absorbed on dry polypropylene beads, manufactured by Chiralvision; and cross-linked aggregate of lipase B from Candida antarctica, manufactured by CLEA. The enzyme may also be a recombinant Candida antarctica lipase B, for example a recombinant Candida antarctica lipase B from Aspergillus oryzae, supplied by Sigma Aldrich (non-immobilised).

The reaction of the mixture of compounds (IIA) and (IIB) is suitably carried out at a temperature in the range of from 15 to 140° C. in order to ensure that reaction rates are significant on the one hand and that the enzyme does not deteriorate with long term use on the other. Preferably the temperature employed is in the range of from 25 to 80° C., more preferably in the range of from 30 to 70° C.

The enzyme-catalysed reaction can be carried out on industrial scale in a number of ways. For example, if a supported enzyme is used the reaction can be carried out batchwise in a single stirred or highly back-mixed tank after which the supported enzyme is separated, e.g. by filtration or the use of hydrocyclones. In such a case the residence time of the reactants and the enzyme in the stirred tank will typically be in the range of up to 24, preferably up to 10, more preferably from 1 to 8 hours, and the amount of enzyme used will preferably be in the range of up to 10%, preferably up to 5% by weight of the weight of the mixture of compounds (IIA) and (IIB). An additional solvent/cosolvent chosen to increase the operational stability of the enzyme, and hence enzyme lifetime (such as hexane, heptane and/or toluene) may be used.

In an alternative preferred embodiment, the enzyme-catalysed reaction may be operated as a continuous or semi-continuous process. For example a mixture comprising the compound of formula (IIA) and the compound of formula (IIB), alkyl alcohol (e.g. n-butanol) and optionally additional solvent/co-solvent (e.g. acetone, toluene, heptane) may be brought into contact with the enzyme (e.g. an immobilised enzyme such as Novozym-435) by passing the mixture through a packed bed of enzyme (e.g. present in a column). In such flow processes, the residence time is selected so as to ensure high conversion. In a particularly preferred embodiment, the packed bed is vertical and the mixture is fed into the top of the column.

In one preferred embodiment, the enzyme-catalysed reaction is carried out continuously in a tower reactor by for example trickling the mixture of the compounds of formula (IIA) and (IIB) and alkyl alcohol, down through a fixed or fluidised bed of the supported enzyme contained therein. A product mixture comprising the compound of formula (I), the compound of formula (IIB), optionally unreacted compound of formula (IIA), and optionally unreacted alcohol can then be recovered from the bottom of the tower. In this arrangement, the contact time of the reactants with the bed is typically in the range of up to 24 hours. Preferably residency/residence times (contact time of the reactants with the bed) are in the range of from 5 minutes to 8 hours, more preferably from 5 minutes to 2 hours. Arrangements of this type permit continuous or semi-continuous generation of product by flow operations.

The compound of formula (I) is preferably separated from the compound of formula (IIB) and optionally any unreacted compound of formula (IIA) by distillation, more preferably by distillation under reduced pressure. For example, butyl R-lactate has a boiling point of 80° C. to 82° C. at 20 mmHg (2700 Pa). Accordingly, where the compound of formula (I) is butyl R-lactate, the compound of formula (I) may for example be separated from the compound of formula (IIB) by fractional distillation at a temperature of from about 60° C. to about 100° C. at a pressure of from about 1000 Pa to about 5000 Pa. In one embodiment, the compound of formula (I) is removed overhead by distillation. In another embodiment, both the compound of formula (I) and the compound of formula (IIB) are removed overhead by distillation (e.g. they are collected as separate overhead product streams, for example at different temperatures and/or pressures. The distillation column (also known as a fractionating column) used must have the necessary number of theoretical plates to perform its function (i.e. to enable separation of the compounds of formula (I) and (IIB)).

The process of the invention allows for the possibility of recovering and recycling reagents, products and catalysts used in the process. For example, the enzyme may be recovered and recycled to the process. Where the enzyme-catalysed reaction is operated in a batch-type reactor, the mixture containing the compound of formula (I) and the compound of formula (IIB) may be separated from the enzyme by, for example, filtration of the enzyme, or by decanting or siphoning off the mixture. Preferably, in the case of a batch-type process, the enzyme is re-used at least once, more preferably at least twice, still more preferably at least 5 times, yet more preferably at least 10 times, most preferably at least 20 times.

In the case of a process where the mixture containing the compounds of formula (IIA) and (IIB) and alkyl alcohol are passed through a packed bed of enzyme (i.e. a continuous or semi-continuous flow process), product and enzyme are continually being separated from one another and the enzyme is continually being recycled. Accordingly, in one preferred embodiment, the process of the invention is a continuous or semi-continuous process which comprises reacting the mixture of the compounds of formula (IIA) and (IIB) with alkyl alcohol in the presence of a Candida antarctica lipase B enzyme to produce a product comprising the compound of formula (I) and the compound of formula (IIB), by passing a solution containing the mixture of the compounds of formula (IIA) and (IIB), alkyl alcohol, and optionally solvent/co-solvent, through a packed bed of immobilised enzyme.

Unreacted alkyl alcohol, and/or optional solvent/co-solvent may also be recovered and recycled. For example, these components may be separated from the products of the process by distillation, and fed back into the process. Alkyl alkanoate produced by the process (produced by enzyme-catalysed reaction of alkyl alcohol with the compound of formula (IIA)) may also be recycled to the process, for example following conversion of the alkyl alkanoate into alkyl alcohol and alkanoic acid, and optional subsequent conversion of the alkanoic acid into the corresponding acid chloride or anhydride if desired.

A particular advantage of the process is that it allows for straightforward recycling of S-lactic acid-based byproducts produced by the process, in particular the compound of formula (IIB) (alkyl α-acyl-S-oxypropionate). For example, the compound of formula (IIB) and the compound of formula (I) may be separated from each other (e.g. by distillation under reduced pressure), the compound of formula (IIB) may then be converted into a mixture of compounds of formula (IIA) and (IIB) by subjecting it to stereoisomerisation conditions, and the mixture recycled to the process, thereby allowing production of additional alkyl R-lactate (the compound of formula (I)). Stereoisomerisation has the effect of randomising the stereochemical configuration at the chiral centre of the O-acyl alkyl lactate. Stereoisomerisation may also be termed racemisation, but is not limited to the production of an optically inactive racemate—in other words, stereoisomerisation can produce a mixture of optical isomers in non-equal amounts.

The compound of formula (IIB) is preferably contacted with a stereoisomerisation catalyst. Catalysts useful for stereoisomerisation of the compound of formula (IIB) include basic catalysts, such as 1,8-diazabicyclo[5.4.0]undec-7-ene (DBU) and 1,8-diaza-[2.2.2]-bicyclooctane (DABCO). In one embodiment, the stereoisomerisation catalyst is DBU. In another embodiment, the stereoisomerisation catalyst is DABCO. In these cases, the stereoisomerisation reaction may for example be carried out at a temperature in the range of from 80 to 220° C., more preferably from about 100 to about 200° C., most preferably from about 120 to about 180° C. Another suitable stereoisomerisation catalyst is a metal alkoxide catalyst, preferably a C₁ to C₈ metal alkoxide, more preferably a sterically hindered metal alkoxide of a tertiary alcohol, such as potassium t-butoxide. Use of sterically hindered alkoxides avoids or at least reduces the extent of transesterification during the stereoisomerisation reaction. Where the stereoisomerisation catalyst is a metal alkoxide, the stereoisomerisation may for example be carried out at a temperature in the range of from 10 to 150° C., more preferably from 25 to 100° C. The stereoisomerisation catalyst may be a Lewis acid, e.g. a metal salt such as a stearate, for example calcium stearate.

In some embodiments, stereoisomerisation of the compound of formula (IIB) may involve contacting the compound with a stereoisomerisation catalyst at elevated temperature. In some embodiments, the temperature and pressure conditions are selected so that the products of the stereoisomerisation reaction (the mixture comprising the compound of formula (IIA) and (IIB)) may be recovered by distillation as they are produced.

Stereoisomerisation of the compound of formula (IIB) directly provides a mixture of compounds (IIA) and (IIB), the feedstock for the stereoselective deacylation process of the invention, and so provides a straightforward process for recycling the S-lactic acid-based byproduct. The compound of formula (IIB) undergoes stereoisomerisation (randomisation of the stereochemical configuration at the chiral centre) under relatively mild conditions, providing a high yield of the stereoisomerisation product (the mixture of compounds of formula (IIA) and (IIB)).

As discussed above, the most readily available source of lactic acid is S-lactic acid. The present inventors have identified a flexible and efficient processing solution which permits the production of R-lactic acid-based materials, and which makes use of a wide variety of comparatively cheap and readily available S-lactic acid-based feedstocks.

The stereoisomerisation reaction described above, in conjunction with the enzyme-catalysed stereoselective deacylation reaction, provides a route for producing R-alkyl lactate from such S-lactic-acid based feedstocks. Accordingly, the invention also provides a process for producing a compound of formula (I) comprising i) subjecting a compound of formula (IIB) to stereoisomerisation conditions to produce a mixture comprising a compound of formula (IIA) and a compound of formula (IIB), and ii) reacting the mixture comprising a compound of formula (IIA) and a compound of formula (IIB) with an alkyl alcohol in the presence of a Candida antarctica lipase B enzyme to produce a product comprising the compound of formula (I) and the compound of formula (IIB).

It will be appreciated that the stereoisomerisation reaction described above may alternatively be carried out using a compound of formula (IIA), i.e. using (R)—O-acyl alkyl lactate to produce the mixture of compounds of formula (IIA) and (IIB), although the use of starting materials derived from R-lactic acid is less preferred. Accordingly the invention also provides a process for producing a compound of formula (I) comprising i) subjecting a compound of formula (IIA) to stereoisomerisation conditions to produce a mixture comprising a compound of formula (IIA) and a compound of formula (IIB), and ii) reacting the mixture comprising a compound of formula (IIA) and a compound of formula (IIB) with an alkyl alcohol in the presence of a Candida antarctica lipase B enzyme to produce a product comprising the compound of formula (I) and the compound of formula (IIB).

The stereoisomerisation reaction may also find use in processes other than those involving stereoselective deacylation of O-acyl alkyl lactates. Accordingly, the invention also provides a process for producing a mixture comprising a compound of formula (IIA) and a compound of formula (IIB) comprising subjecting a compound of formula (IIA) to stereoisomerisation conditions to produce the mixture comprising the compound of formula (IIA) and the compound of formula (IIB). The invention also provides a process for producing a mixture comprising a compound of formula (IIA) and a compound of formula (IIB) comprising subjecting a compound of formula (IIB) to stereoisomerisation conditions to produce the mixture comprising the compound of formula (IIA) and the compound of formula (IIB).

S-lactic acid-based materials other than the compound of formula (IIB) (alkyl α-acyl-S-oxypropionate) may also find use as feedstocks for the process of the invention, by subjecting them to stereoisomerisation conditions, and converting the product into a mixture of compounds of formula (IIA) and (IIB). Accordingly, the invention also provides a process for producing a compound of formula (I)

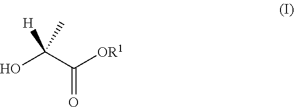

comprising i) subjecting an initial compound comprising substructure (III)

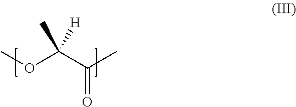

to stereoisomerisation conditions; ii) if necessary, converting at least a portion of the product of step i) into a mixture comprising a compound of formula (IIA) and a compound of formula (IIB); and

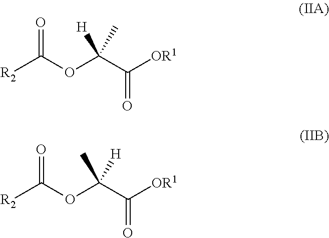

iii) reacting the mixture comprising a compound of formula (IIA) and a compound of formula (IIB) with an alkyl alcohol in the presence of a Candida antarctica lipase B enzyme to produce a product comprising the compound of formula (I) and the compound of formula (IIB); wherein R¹ is C₁ to C₆ alkyl; and wherein R² is H or C₁ to C₁₂ alkyl.

A variety of lactic acid-based building blocks may be used as feedstocks, including waste materials, byproducts or polylactic acid from other lactic acid-based processes. The initial compound comprises substructure (III) i.e. the initial compound comprises a building block based on S-lactic acid. For example, the initial compound may be oligomeric S-lactic acid, S-lactic acid or a salt thereof (e.g. ammonium S-lactate), alkyl S-lactate, alkyl S,S-lactyllactate, S,S-lactide and/or poly S-lactic acid (e.g. recycled poly S-lactic acid). Any of those materials mixed with corresponding R-materials (R-lactic acid or a salt thereof, alkyl R-lactate, alkyl R,R-lactyllactate, R,R-lactide, oligomeric R-lactic acid, poly R-lactic acid) may also be used as feedstocks. Thus for example, waste lactic acid feedstocks containing S-lactic acid as a major constituent together with R-lactic acid as a minor constituent may be used.

Similarly, oligomeric lactic acid comprising a majority of S-lactic acid monomer units and a minority of R-lactic acid monomer units may be utilised. Thus, the initial compound may, in addition to comprising substructure (III), comprise substructure (IV),

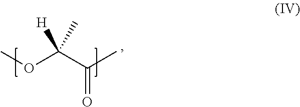

i.e. the initial compound may also comprise a building block based on R-lactic acid. As discussed above, the initial compound may comprise an oligomer of lactic acid having both R- and S-lactic acid monomer units. Other examples include alkyl lactyllactate having both R- and S-chiral centres, and R,S-lactide. Polylactic acid (e.g. waste poly S-lactic acid of insufficient chiral purity to be useful as a commercial product, or recycled polylactic acid), may be used. A single initial compound or a combination of initial compounds may be used as the feedstock, optionally together with R-lactic acid based materials as discussed above. For example, possible feedstocks include polylactic acid plant waste streams (containing, e.g. lactide monomers (mixtures of R,R- S,S- and/or R,S-lactide); mixtures of oligomeric S-lactic acid, oligomeric R-lactic acid and/or oligomer of lactic acid having both S- and R-lactic acid monomer units), as well as stereocomplex polylactic acid.

Subjecting an initial compound to stereoisomerisation conditions has the effect of randomising the stereochemical configuration at one or more chiral centres within the compound comprising substructure (I). For example, when the initial compound is alkyl S-lactate, subjecting alkyl S-lactate to stereoisomerisation conditions leads to the production of a mixture of alkyl R-lactate and alkyl S-lactate. When the initial compound is S-lactic acid, subjecting the S-lactic acid to stereoisomerisation conditions leads to the production of a mixture of R-lactic acid and S-lactic acid. When the initial compound is oligomeric S-lactic acid, subjecting the oligomeric S-lactic acid to stereoisomerisation conditions leads to the production of oligomeric lactic acid (i.e. oligomer of lactic acid comprising both R- and S-lactic acid monomer units). When the initial compound is alkyl S,S-lactyllactate, subjecting the alkyl S,S-lactyllactate to stereoisomerisation conditions leads to the production of alkyl lactyllactate (i.e. a mixture comprising alkyl R,R-lactyllactate, alkyl S,S-lactyllactate, alkyl R,S-lactyllactate and alkyl S,R-lactyllactate). When the initial compound is R,S-lactide, subjecting the R,S-lactide to stereoisomerisation conditions leads to the production of a mixture of R,R-, S,S- and R,S-lactide. In other words, subjecting the initial compound comprising substructure (III) to stereoisomerisation conditions results in a change from a first stereoisomeric distribution to a second different stereoisomeric distribution.

The initial compound is preferably contacted with a stereoisomerisation catalyst. Catalysts useful for stereoisomerisation of compounds comprising substructure (III) include basic catalysts, which are preferred, and acidic catalysts. Examples of basic catalysts include 1,8-diazabicyclo[5.4.0]undec-7-ene (DBU) and 1,8-diaza-[2.2.2]-bicyclooctane (DABCO). A preferred stereoisomerisation catalyst is an amine catalyst, preferably a tertiary amine catalyst, more preferably a trialkylamine wherein each alkyl group independently comprises 2 to 12 carbon atoms, most preferably triethylamine. Other examples of preferred amine catalysts include ammonia, trimethylamine, tri(n-propyl)amine, tri(n-butyl)amine, tri(n-pentyl)amine, tri(n-hexyl)amine, tri(n-octyl)amine, tri(n-decyl)amine, Alamine 336, pyridine, lutidine, dimethylamine, diethylamine, di(n-propyl)amine, di(iso-propyl)amine, di(n-butyl)amine, di(tert-butylamine), diphenylamine, methylamine, ethylamine, n-propylamine, iso-propylamine, n-butylamine, sec-butylamine, iso-butylamine, tert-butylamine, n-pentylamine, n-hexylamine, 2-ethylhexylamine, n-octylamine, n-decylamine, phenylamine (aniline), benzylamine. Such amine catalysts are particularly suitable for use where the initial compound comprises oligomeric S-lactic acid, oligomeric lactic acid having both R- and S-lactic acid monomer units, alkyl S-lactate, alkyl S,S-lactyllactate, alkyl S,R-lactyllactate, alkyl R,S-lactyllactate, S,S-lactide and/or R,S-lactide. Such amine catalysts are also suitable for use with feedstocks comprising one or more of those initial compounds together with one or more of the corresponding R-materials (e.g. oligomeric R-lactic acid, alkyl R-lactate, alkyl R,R-lactyllactate, R,R-lactide). The amine-catalysed stereoisomerisation reaction can be carried out at any temperature suitable to effect randomisation of chiral centres within the compound having substructure (III), for example it may be carried out at a temperature of from about 80 to about 220° C., more preferably from about 100 to about 200° C., most preferably from about 120 to about 180° C.

Another catalyst useful for stereoisomerisation of compounds comprising substructure (III) is a metal alkoxide catalyst, preferably a C₁ to C₈ metal alkoxide, more preferably sodium n-butoxide, sodium t-butoxide or potassium t-butoxide. Metal alkoxides are particularly suitable for use with initial compounds such as alkyl S-lactate (e.g. where the feedstock is alkyl S-lactate, or a mixture containing alkyl S-lactate as the major component and alkyl R-lactate as the minor component). In that case, the metal alkoxide is preferably selected so that the alcohol from which the alkoxide is derived corresponds to the alcohol from which the lactate ester is derived, so that any transesterification that may take place does not result in the formation of mixtures of alkyl lactates having different ester groups (e.g where the lactate ester is the n-butyl ester, the metal alkoxide is preferably sodium n-butoxide). In some circumstances, metal alkoxides such as sodium n-butoxide may also be preferred for use with initial compounds where simultaneous alcoholysis and randomisation of chiral centres is desired, for example with alkyl S,S-lactyllactate as the initial compound. Alternatively, where it is desired to reduce or avoid transesterification and/or alcoholysis, a sterically hindered metal alkoxide may be employed, such as potassium or sodium t-butoxide. Where the stereoisomerisation catalyst is a metal alkoxide, the stereoisomerisation may be carried out at any temperature suitable to effect randomisation of chiral centres within the compound having substructure (III), for example it may be carried out at a temperature of from 25to 150° C., more preferably from 50 to 100° C. Further examples of suitable catalysts include metal salts such as metal stearates, preferably calcium stearate. Where the initial compound is lactic acid, the catalyst may be a metal hydroxide, such as sodium hydroxide.

In some embodiments, where the initial compound is S-lactic acid (e.g. the feedstock may be S-lactic acid, or a mixture of S-lactic acid as the major component and R-lactic acid as the minor component), suitable stereoisomerisation conditions may involve exposing the initial compound to high temperature (e.g. from about 250 to about 325° C.) and high pressure conditions (e.g about 150 bar (15,000,000 Pa)), preferably in the presence of a metal hydroxide catalyst, such as sodium hydroxide.

If necessary, at least a portion of the product of step i) is converted into a a mixture comprising a compound of formula (IIA) and a compound of formula (IIB) (i.e. where the initial compound was not a compound of formula (IIA) or (IIB) and the product of step i) is not already a mixture comprising compounds (IIA) and (IIB)). This may be achieved by routine methods. For example, where the initial compound is alkyl S-lactate and the product of step i) is a mixure of alkyl S-lactate and alkyl R-lactate, the alkyl S-lactate and alkyl R-lactate may for example be converted into the mixture comprising compounds (IIA) and (IIB) by reaction of the alcohol moiety with a carboxylic acid anhydride (e.g. acetic anhydride) or an acid chloride (e.g. acetyl chloride). In a further example, where the initial compound is alkyl S-lactate and the product of step i) is a mixure of alkyl S-lactate and alkyl R-lactate, the alkyl S-lactate and alkyl R-lactate may for example be converted into the mixture comprising compounds (IIA) and (IIB) by reaction of the alcohol moiety with a carboxylic acid (e.g. acetic acid), preferably in the presence of an acidic catalyst (e.g. Amberlyst 15 hydrogen form). Where the initial compound is oligomeric S-lactic acid and the product of step i) is oligomer of lactic acid having both R- and S-lactic acid monomer units, the oligomer of lactic acid may for example be converted into the mixture comprising compounds (IIA) and (IIB) by alcoholysis with alkyl alcohol to produce a mixture comprising alkyl R-lactate and alkyl S-lactate, and reacting the mixture of alkyl R-lactate and alkyl S-lactate with a carboxylic acid anhydride (e.g. acetic anhydride) or an acid chloride (e.g. acetyl chloride). In a further example, where the initial compound is oligomeric S-lactic acid and the product of step i) is oligomer of lactic acid having both R- and S-lactic acid monomer units, the oligomer of lactic acid may for example be converted into the mixture comprising compounds (IIA) and (IIB) by alcoholysis with alkyl alcohol to produce a mixture comprising alkyl R-lactate and alkyl S-lactate, and reacting the mixture of alkyl R-lactate and alkyl S-lactate with a carboxylic acid (e.g. acetic acid) in the presence of an acidic catalyst (e.g. Amberlyst 15 hydrogen form). The use of compounds such as carboxylic acids and catalytic quantities of acid catalysts provides a process which generates byproducts having low toxicity, and which is particularly amenable to commodity scale manufacturing.

As discussed above, the compound of formula (IIB) may also be subjected to stereoisomerisation conditions, in which case the mixture of compounds (IIA) and (IIB) may be obtained directly from step i) of the process.

It will be understood that a process based on using an R-lactic acid-based feedstock, rather than an S-lactic acid-based feedstock, can also provide useful products, i.e. products containing the S-lactic acid building block (e.g. the compound of formula (IIB) (S)—O-acyl alkyl lactate). Thus, in another aspect, the invention provides a process for producing a compound of formula (IIB)

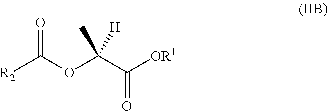

comprising i) subjecting an initial compound comprising substructure (IV)

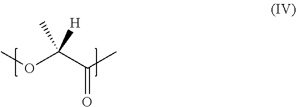

to stereoisomerisation conditions; ii) if necessary, converting at least a portion of the product of step i) into a mixture comprising a compound of formula (IIA) and a compound of formula (IIB); and

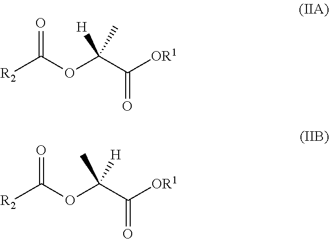

iii) reacting the mixture comprising a compound of formula (IIA) and a compound of formula (IIB) with an alkyl alcohol in the presence of a Candida antarctica lipase B enzyme to produce a product comprising a compound of formula (I)

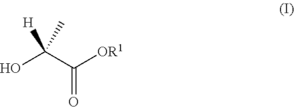

and the compound of formula (IIB); wherein R¹ is C₁ to C₆ alkyl; and wherein R² is H or C₁ to C₁₂ alkyl. In that process, the initial compound may for example be poly-R-lactic acid, oligomeric R-lactic acid, R-lactic acid or a salt thereof, alkyl R-lactate, alkyl R,R-lactyllactate and/or R,R-lactide.

The compound of formula (I) (alkyl R-lactate) produced by the process of the invention can be converted into further useful downstream products by routine methods. Accordingly, the invention provides a process for producing R-lactic acid, oligomeric R-lactic acid, R,R-lactide and/or poly R-lactic acid, comprising producing the compound of formula (I) by a process according to the invention, and converting the compound of formula (I) into R-lactic acid, oligomeric R-lactic acid, R,R-lactide and/or poly-R-lactic acid. The alkyl R-lactate may be converted into those products directly or indirectly. By way of example, alkyl R-lactate may be converted into R-lactic acid by hydrolysis of the alkyl ester group. In that case, the alkyl alcohol produced may be recovered and recycled to the process. Alkyl R-lactate may also be converted to oligomeric R-lactic acid, for example by heating in the presence of a transesterification catalyst (such as titanium (IV) tetra(iso-propoxide) or tin(II)bis(2-ethyl hexanoate)) and removing the alcohol produced, or by first converting the alkyl R-lactate to R-lactic acid, and by heating the R-lactic acid and removing the water that is produced. Alkyl R-lactate may also be converted to R,R-lactide, for example by producing oligomeric R-lactic acid, and by heating the oligomeric R-lactic acid in the presence of a transesterification catalyst (e.g. a Lewis acid catalyst comprising an oxide, alkoxide or carboxylate salt of a metal). An advantage of processes involving subsequent conversion into R,R-lactide is that the production of alkyl R-lactate having lower enantiomeric excess can be tolerated. In such cases conversion into R,R-lactide will also result in the formation of R,S-lactide as a minor product (and very small quantities of S,S lactide). The R,S-lactide produced may readily be separated from the R,R-lactide, for example by distillation and/or crystallisation and/or washing. Alkyl R-lactate may also be converted into poly R-lactic acid, for example by producing R,R-lactide, and by polymerising R,R-lactide by contacting it with a suitable catalyst.

The invention also provides a process for producing stereocomplex polylactic acid, comprising producing poly-R-lactic acid by a process according to the invention, and combining the poly-R-lactic acid with poly-S-lactic acid, for example using melt blending, to produce stereocomplex polylactic acid.

The other principal lactate product produced by the process of the invention is (S)—O-acyl alkyl lactate (the compound of formula IIB), which can also be converted into further useful downstream products by routine methods. Accordingly, the invention provides a process for producing alkyl S-lactate, S-lactic acid, oligomeric S-lactic acid, S,S-lactide or poly S-lactic acid, comprising producing the compound of formula (IIB) by a process according to the invention, and converting the compound of formula (IIB) into alkyl S-lactate, S-lactic acid, oligomeric S-lactic acid, S,S-lactide and/or poly-S-lactic acid. The compound of formula (IIB) may be converted into those products directly or indirectly.

The invention also provides a process for producing stereocomplex polylactic acid, comprising producing poly-S-lactic acid by a process according to the invention, and combining the poly-S-lactic acid with poly-R-lactic acid, for example using melt blending, to produce stereocomplex polylactic acid.

The following examples illustrate the invention.

### EXAMPLE 1

### Batch Solvent Free Enantioseparation of racemic O-acetyl-(n-butyl)lactate

A racemic mixture of O-acetyl-(n-butyl)lactate (1.0 g, 5.32 mmol), n-butanol (0.39 g, 5.27 mmol, 1 equivalent) and Novozym-435 (50 mg, 5% by weight of O-acetyl-(n-butyl)lactate) were charged to a 15 ml Eppendorf tube and shaken at 550 rpm, 50° C. for 23 h. Samples of the reaction mixture were analysed for butyl R-lactate at 4 and 23 hours by quantitative chiral gas chromatography.

| Time (h): | % Conversion*: | Enantiomeric excess: |
|---|---|---|
| 4 | 53 | 100 |
| 23 | 91 | 99 |

(*based on theoretical conversion of (R)—O-acetyl-(n-butyl)lactate to n-butyl R-lactate)

The results demonstrate that stereoselective deacylation of (R)—O-acetyl-(n-butyl)-lactate to butyl R-lactate takes place without significant deacylation of (S)—O-acetyl-(n-butyl)-lactate being observed.

### EXAMPLE 2

### Batch Solvent Free Enantioseparation of racemic 0-butanoyl-(n-butyl)lactate(butyl butyryl lactate)

A racemic mixture of O-butanoyl-(n-butyl)lactate (2.0 g, 9.26 mmol), n-butanol (2.06 g, 3 equivalents) and Novozym-435 (100 mg, 5% by weight of O-butanoyl-(n-butyl)lactate) were charged to a 15 ml Eppendorf tube and shaken at 650 rpm, 55° C. for 21 h. Samples of the reaction mixture were analysed for butyl R-lactate at 1, 3 and 21 hours by quantitative chiral gas chromatography.

| Time (h): | % Conversion*: | Enantiomeric excess: |
|---|---|---|
| 1 | 60 | 98 |
| 3 | 90 | 98 |
| 23 | 99 | 97 |

(*based on theoretical conversion of (R)—O-butanoyl-(n-butyl)lactate to n-butyl R-lactate)

The results demonstrate that stereoselective deacylation of (R)—O-butanoyl-(n-butyl)lactate butyl R-lactate takes place without significant deacylation of (S)—O-butanoyl-(n-butyl)lactate being observed.

### EXAMPLE 3

### Batch Enantioseparation of racemic O-propanoyl-(n-butyl)lactate Using heptane as Solvent

A racemic mixture of crude rac-O-propanoyl(n-butyl)lactate in heptane (which contained approximately 4% rac-butyllactate), n butanol and Novozym-435 was charged to a 15 ml Eppendorf tube and shaken at 650 rpm, 55° C. for 18 h. Samples of the reaction mixture were analysed for butyl R-lactate at 1 h, and 18 h by chiral gas chromatography.

| Time (h): | Enantiomeric excess: |
|---|---|
| 1 | 75 |
| 18 | 91 |

Despite the presence of (S)-butyllactate in the starting mixture, the results demonstrate that stereoselective deacylation of (R)—O-propanoyl(n-butyl)lactate to butyl R-lactate takes place without significant deacylation of (S)—O-propanoyl(n-butyl)lactate as the amount of S-butyllactate was not observed to increase above that already contained in the starting material.

### EXAMPLE 4

### Batch Enantioseparation of Racemic O-methanoyl(n-butyl)lactate Under Solvent Free Conditions at 50° C.

A racemic mixture of distilled rac-O-methanoyl(n-butyl)lactate (1.0 g, 5.75 mmol) n-butanol (0.43 g, 1 equivalent) and Novozym-435 (50 mg, 5% by weight of O-methanoyl(n-butyl)lactate) was charged to a 15 ml Eppendorf tube and shaken at 750 rpm, 50° C. for 18 h. Samples of the reaction mixture were analysed for butyl R-lactate at 1 h, 3 h and 22 h by quantitative chiral gas chromatography.

| Time (h): | % Conversion*: | Enantiomeric excess: |
|---|---|---|
| 1 | 66 | 94 |
| 3 | 90 | 92 |
| 22 | 97 | 61 |

(*based on theoretical conversion of (R)—O-methanoyl-(n-butyl)lactate to butyl R-lactate)

These results indicate a rapid stereoselective deacylation of (R)—O-methanoyl-(n-butyl)lactate takes place. Stopping the reaction at 3 hours produces both reaction products in 92% ee. Allowing the reaction to progress further gave (S)—O-methanoyl-(n-butyl)lactate at 95% ee and butyl-R-lactate at 61% ee.

### EXAMPLE 5

### Batch Enantioseparation of Racemic O-methanoyl(n-butyl)lactate Under Solvent Free Conditions at 20° C.

A racemic mixture of distilled rac-O-methanoyl(n-butyl)lactate (2.5 g, 14.37 mmol), n-butanol (2.63 g, 2 equivalent) and Novozym-435 (104 mg, 4% by weight of O-methanoyl(n-butyl)lactate) was charged to a 15 ml Eppendorf tube and shaken at 750 rpm, 20° C. for 48 h. Samples of the reaction mixture were analysed for butyl R-lactate at 2 h, 4 h, 6 h, 8 h and 24 h by quantitative chiral gas chromatography.

| Time (h): | % Conversion*: | Enantiomeric excess: |
|---|---|---|
| 2 | 30 | 96 |
| 4 | 51 | 97 |
| 6 | 67 | 97 |
| 8 | 77 | 97 |
| 24 | 96 | 94 |

(*based on theoretical conversion of (R)—O-methanoyl-(n-butyl)lactate to butyl R-lactate)

These results indicate a rapid stereoselective deacylation of (R)—O-methanoyl-(n-butyl)lactate takes place. Stopping the reaction at 8 hours produces butyl-R-lactate with 97% ee in 77% solution yield. Allowing the reaction to progress further to 24 hours gave (S)—O-methanoyl-(n-butyl)lactate at 97% ee and butyl-R-lactate at 94% ee

### EXAMPLE 6

### Parallel Batch Enantioseparation of Racemic O-acylated-(n-butyl)lactates Under Solvent Free Conditions at 55° C.

A mixture of the respective distilled rac-O-acyl(n-butyl)lactate ((i) rac-O-methanoyl(n-butyl)lactate, (ii) rac-O-acetyl-(n-butyl)lactate, (iii) rac-O-Butanoyl-(n-butyl)lactate) (2.5 g), n-butanol (2.0 molar equivalents) and Novozym-435 (4.6×10⁻³ mol of Candida antarctica lipase B, based on 21% by weight of Novozym-435 being Candida antarctica lipase B) was charged to a 15 ml Eppendorf tube and shaken at 600 rpm, 55° C. for 23 h. Samples of the reaction mixture were analysed for butyl R-lactate at the indicated timepoints by quantitative chiral gas chromatography.

### (i) rac-O-Methanoyl (n-butyl)lactate

| Time (h): | % Conversion*: | Enantiomeric excess %: |
|---|---|---|
| 0 | 0 | 0 |
| 1 | 79 | 94 |
| 2 | 92 | 92 |
| 3 | 96 | 89 |

(*based on theoretical conversion of (R)—O-methanoyl-(n-butyl)lactate to butyl R-lactate)

### (ii) rac-O-Acetyl-(n-butyl)lactate

| Time (h): | % Conversion*: | Enantiomeric excess %: |
|---|---|---|
| 0 | 0 | 0 |
| 1 | 22 | 100 |
| 2 | 36 | 100 |
| 3 | 48 | 100 |
| 5 | 64 | 100 |
| 23 | 97 | 98 |

(*based on theoretical conversion of (R)—O-acetyl-(n-butyl)lactate to butyl R-lactate)

### (iii) rac-O-n-Butanoyl-(n-butyl)lactate(butyl butyryl lactate)

| Time (h): | % Conversion*: | Enantiomeric excess %: |
|---|---|---|
| 0 | 0 | 0 |
| 1 | 50 | 100 |
| 2 | 72 | 100 |
| 3 | 88 | 99 |
| 5 | 94 | 99 |
| 23 | 99 | 98 |

(*based on theoretical conversion of (R)—O-n-butanoyl-(n-butyl)lactate to butyl R-lactate)

These results indicate that a rapid stereoselective deacylation of (R)—O-acyl-(n-butyl)lactates takes place.

### EXAMPLE 7

### Racemisation of (S)—O-acetyl-(n-butyl)lactate using DBU

(S)—O-acetyl-(n-butyl)lactate (2.5 g, 13.3 mmol) and DBU (10 mole %) were stirred at 150° C. for 22 hours. Samples of the reaction mixture at 3 and 22 hours were analysed by quantitative chiral gas chromatography confirming that complete racemisation had occurred by 3 hours, along with the formation of some racemic n-butyl lactate (6% after 3 hours; 12% after 22 hours) due to deacylation.

The results demonstrate that racemisation of (S)—O-acetyl-(n-butyl)lactate can be achieved with an amine base, allowing recycle of racemic O-acetyl-(n-butyl)-lactate to the process. Distillation of the product mixture could be used to remove any racemic butyllactate formed in the racemisation process.

### EXAMPLE 8

### Racemisation of (S)—O-acetyl-(n-butyl)lactate Using Potassium t-butoxide

An (S)—O-acetyl-(n-butyl)lactate solution (2.5 g, 13.3 mmol) in THF and potassium t-butoxide (10 mole %) was charged to a 15 ml Eppendorf tube and stirred at room temperature for 17 hours. Samples of the reaction mixture at 17 hours were analysed by quantitative chiral gas chromatography confirming that complete racemisation had occurred, along with the formation of some racemic n-butyl lactate (12%) due to deacylation.

The results demonstrate that racemisation of (S)—O-acetyl-(n-butyl)lactate can be achieved with potassium t-butoxide, allowing recycle of racemic O-acetyl-(n-butyl)-lactate to the process. Distillation of the product mixture could be used to remove any racemic n-butyllactate formed in the racemisation process.

### EXAMPLE 9

### Production of Racemic O-acetyl-(n-butyl)lactate from Racemic n-butyllactate Using Acetic Acid and Amberlyst 15 Hydrogen Form

Glacial acetic acid (5.2 g, 6.5 equivalents with respect to n-butyllactate) was refluxed with rac-n-butyllactate (1.97 g) i) in the presence and ii) in the absence of Amberlyst 15 hydrogen form (0.5 g). In the absence of Amberlyst 15 hydrogen form, ˜70% of the racemic O-acetyl-(n-butyl)lactate product was produced after 22 h reflux, as determined by chiral GC. In contrast, the presence of Amberlyst 15 hydrogen form effectively catalysed the reaction, producing ˜88% of the racemic O-acetyl-(n-butyl)lactate product after only 2 h reflux, as determined by chiral GC. No further reaction was seen after 2 h suggesting that an equilibrium was reached. The product mixture was partitioned with heptanes, and washed with water and aqueous NaHCO₃ solution to neutralise and/or remove most of the unconverted racemic-butyllactate and acetic acid. This upgraded the product to 96% purity with only 4% of rac-n-butyllactate remaining.

1. A process for producing a compound of formula (I) 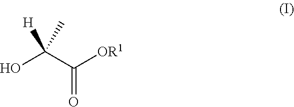
comprising reacting a mixture comprising a compound of formula (IIA) and a compound of formula (IIB) 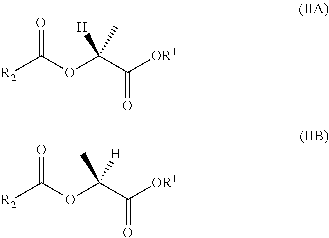
with an alkyl alcohol in the presence of a Candida antarctica lipase B enzyme to produce a product comprising the compound of formula (I) and the compound of formula (IIB); wherein R¹ is C₁ to C₆ alkyl; and wherein R² is H or C₁ to C₁₂ alkyl.

2. A process as claimed in claim 1, wherein
a) the compound of formula (I) and the compound of formula (IIB) are separated;
b) the compound of formula (IIB) is subjected to stereoisomerisation conditions thereby producing a mixture of the compound of formula (IIA) and the compound of formula (IIB); and
c) the mixture of the compound of formula (IIA) and the compound of formula (IIB) is recycled to the process.

3. A process as claimed in claim 2, wherein in step b) the compound of formula (IIB) is contacted with a stereoisomerisation catalyst.

4. A process as claimed in claim 1, wherein the compound of formula (I) is separated from the compound of formula (IIB) by distillation.

5. A process as claimed in claim 1, wherein R¹ is n-butyl.

6. A process as claimed in claim 1, wherein R² is ethyl or n-propyl.

7. A process as claimed in claim 1, wherein the alkyl alcohol has the formula R¹—OH, and wherein the alkyl alcohol R¹ group is the same as the R¹ group in the compounds of formula (IIA) and (IIB).

8. A process as claimed in claim 1, wherein the molar ratio of alkyl alcohol to the compound of formula (IIA) is in the range of from 0.8:1 to 10:1.

9. A process as claimed in claim 1, wherein the mixture comprising the compound of formula (IIA) and the compound of formula (IIB) is reacted with alkyl alcohol at a temperature of from 25° C. to 80° C.

10. A process as claimed in claim 1, wherein the enzyme is chemically or physically immobilised.

11. A process as claimed in claim 1, wherein a mixture comprising the compound of formula (IIA), the compound of formula (IIB) and alkyl alcohol is passed through a packed bed of enzyme.

12. A process as claimed in claim 1, comprising the preceding steps of:
i) subjecting an initial compound comprising substructure (III) 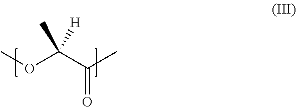
to stereoisomerisation conditions; and
ii) if necessary, converting at least a portion of the product of step i) into a mixture comprising a compound of formula (IIA) and a compound of formula (IIB).

13. A process as claimed in claim 12, wherein the mixture comprising the compound of formula (IIA) and the compound of formula (IIB) is produced by subjecting a compound of formula (IIB) to stereoisomerisation conditions.

14. A process for producing R-lactic acid, oligomeric R-lactic acid, R,R-lactide and/or poly R-lactic acid, comprising producing the compound of formula (I) by a process according to claim 1, and converting the compound of formula (I) into R-lactic acid, oligomeric R-lactic acid, R,R-lactide and/or poly R-lactic acid.

15. A process for producing alkyl S-lactate, S-lactic acid, oligomeric S-lactic acid, S,S-lactide or poly S-lactic acid, comprising producing the compound of formula (IIB) by a process according to claim 1, and converting the compound of formula (IIB) into alkyl S-lactate, S-lactic acid, oligomeric S-lactic acid, S,S-lactide and/or poly-S-lactic acid.

16. A process for producing stereocomplex polylactic acid, comprising producing poly-R-lactic acid according to claim 14, and combining the poly-R-lactic acid with poly-S-lactic acid to produce stereocomplex polylactic acid.

17. A process for producing stereocomplex polylactic acid, comprising producing poly-S-lactic acid according to claim 15, and combining the poly-S-lactic acid with poly-R-lactic acid to produce stereocomplex polylactic acid.
